(19) United States
(12) Patent Application Publication
(10) Pub. No.: US 2016/0367193 A1
(43) Pub. Date: Dec. 22, 2016

(54) REMOTE CONTROLLER AND HEALTH DETECTION SYSTEM

(71) Applicant: BOE TECHNOLOGY GROUP CO., LTD., Beijing (CN)

(72) Inventor: Jingyu ZHANG, Beijing (CN)

(73) Assignee: BOE TECHNOLOGY GROUP CO., LTD., Beijing (CN)

(21) Appl. No.: 14/905,935

(22) PCT Filed: Jul. 24, 2015
(86) PCT No.: PCT/CN2015/085021

§ 371 Date: Jan. 18, 2016

(30) Foreign Application Priority Data
Feb. 28, 2015 (CN) 201510091518.5

(51) Int. Cl. A61B 5/00; A61B 5/103; A61B 5/021; G06T 7/00; A61B 5/0402

(52) U.S. Cl. CPC A61B 5/6887; G06T 7/0012; A61B 5/0077; A61B 5/0402; A61B 5/02125; A61B 5/1032; A61B 5/742; A61B 5/0013; G06T 2207/30048; G06T 2207/30088; A61B 2562/0233

(57) ABSTRACT

The present disclosure provides a remote controller and a health detection system. The remote controller includes a main body, a display module, and a health detection module arranged on the main body. The health detection module is configured to detect a health value of a user, and the health value at least includes a heart rate. The display module is connected to the health detection module and configured to display the health value.

### CROSS-REFERENCE TO RELATED APPLICATION

The present application claims a priority of the Chinese patent application No. 201510091518.5 filed on Feb. 28, 2015, which is incorporated herein by reference in its entirety.

### TECHNICAL FIELD

The present disclosure relates to the field of electronic information technology, in particular to a remote controller and a health detection system.

### BACKGROUND

Along with the improvement of the living standards, people pay more and more attention to health. When people want to know their health conditions, a health detection device may be used.

In the related art, when a user wants to know his health condition, he needs to move the health detection device to be in the vicinity of a television, connect the health detection device to a remote controller for the television, and set in the remote controller a correspondence between the health detection device and the remote controller. Then, the user's health condition may be acquired by the health detection device. After the acquisition of the health condition, a health value is generated in accordance with the health condition, and transmitted to the television via the remote controller corresponding to the health detection device, so as to display the health value by the television.

In the related art, the health condition may be detected by the user only when he moves the health detection device to be in the vicinity of the television and connects the remote controller to the health detection device, so this procedure is complex.

### SUMMARY

An object of the present disclosure is to provide a remote controller and a health detection system, so as to simplify a procedure for the health detection.

In one aspect, the present disclosure provides in some embodiments a remote controller, including a main body, a display module, and a health detection module arranged on the main body. The health detection module is configured to detect a health value of a user, and the health value at least includes a heart rate. The display module is connected to the health detection module and configured to display the health value.

Alternatively, the health detection module includes a first heart rate detection unit and a first heart rate processing unit connected to the first heart rate detection unit. The first heart rate detection unit includes a metal button and a metal plate. The first heart rate processing unit is configured to, when a first body part of the user is in contact with the metal button and a second body part of the user is in contact with the metal plate, acquire a potential at the first body part through the metal button, acquire a potential at the second body part through the metal plate, calculate a potential difference between the first body part and the second body part in accordance with the potential at the first body part and the potential at the second body part, and calculate the heart value in accordance with a correspondence between the potential difference and the heart rate as well as the potential difference. The second body part is different from the first body part.

Alternatively, the health detection module includes a second heart rate detection unit and a second heart rate processing unit connected to the second heart rate detection unit. The second heart rate detection unit includes a camera configured to acquire a skin image of the user and transmit the acquired skin image to the second heart rate processing unit. The second heart rate processing unit is configured to calculate the heart rate in accordance with the skin image using an image information processing technique and an image-heart rate algorithm.

Alternatively, the health detection module includes a third heart rate detection unit and a third heart rate processing unit connected to the third heart rate detection unit. The third heart rate detection unit includes a light-emitting diode (LED), and a phototransistor configured to, when a light beam from the LED is reflected by a hand of the user to the phototransistor, generate detection information in accordance with the reflected light beam, and transmit the detection information to the third heart rate processing unit so that the third heart rate processing unit generates the heart rate in accordance with the detection information.

Alternatively, the health value further includes a blood pressure. The health detection module includes a first blood pressure detection unit and a first blood pressure processing unit connected to the first blood pressure detection unit. The first blood pressure detection unit includes a piezoelectric sensor secured underneath a button of the main body of the remote controller. The piezoelectric sensor is configured to, when the button is pressed by the user continuously, collect a pulse signal generated every time the button is pressed, and transmit the collected pulse signal to the first blood pressure processing unit. The first blood pressure processing unit is configured to generate a time difference between the adjacent pulse signals, and calculate the blood pressure in accordance with a correspondence between the time difference and the blood pressure as well as the time difference.

Alternatively, the health value further includes a blood pressure, and the health detection module further includes a second blood pressure processing unit connected to the camera. The camera is further configured to transmit the skin image to the second blood pressure processing unit, so that the second blood pressure processing unit calculates the blood pressure in accordance with the skin image using the image information processing technique and an image-blood pressure algorithm.

Alternatively, the remote controller further includes a health indication module connected to the health detection module and configured to, when the health value exceeds a predetermined range, send a prompt.

Alternatively, the health indication module includes a lamp connected to the health detection module and flickering when the health value exceeds the predetermined range.

Alternatively, the display module is configured to display the health value on the remote controller, or transmit the health value to a television so as to display the health value thereon.

In another aspect, the present disclosure provides in some embodiments a health detection system, including a television and the above-mentioned remote controller.

Alternatively, the remote controller is configured to transmit a detected health value to the television, and the television is configured to display the health value.

Alternatively, the television is further configured to store therein the health value.

According to the remote controller and the health detection system in the embodiments of the present disclosure, the remote controller includes the main body, the display module and the health detection module arranged on the main body. The health detection module is configured to detect the health value of the user, and the display module is connected to the health detection module and configured to display the health value. When the user wants to detect his health condition, the remote controller may be directly used by the user to detect and display the health value, and the user may know his health condition in accordance with the health value. In this way, it is unnecessary for the user to move the health detection device to be in the vicinity of the television and connect the health detection device to the remote controller. As a result, it is able to simplify the procedure for the health detection.

It should be appreciated that, the above and the following descriptions are merely for illustrative purposes, but shall not be used to limit the present disclosure.

### BRIEF DESCRIPTION OF THE DRAWINGS

In order to illustrate the technical solutions of the present disclosure or the related art in a clearer manner, the drawings desired for the present disclosure or the related art will be described hereinafter briefly. Obviously, the following drawings merely relate to some embodiments of the present disclosure, and based on these drawings, a person skilled in the art may obtain the other drawings without any creative effort.

FIG. 1 is a schematic view showing a remote controller according to one embodiment of the present disclosure;

FIG. 2 is a schematic view showing a further remote controller according to one embodiment of the present disclosure;

FIG. 3 is a schematic view showing the structure of the further remote controller in FIG. 2 according to one embodiment of the present disclosure;

FIG. 4 is another schematic view showing the structure of the further remote controller according to one embodiment of the present disclosure;

FIG. 5 is yet another schematic view showing the structure of the further remote controller according to one embodiment of the present disclosure;

FIG. 6 is still yet another schematic view showing the structure of the further remote controller according to one embodiment of the present disclosure;

FIG. 7 is still yet another schematic view showing the structure of the further remote controller according to one embodiment of the present disclosure; and

FIG. 8 is a schematic view showing a health detection system according to one embodiment of the present disclosure.

The above drawings relate to some specific embodiments of the present disclosure, and these embodiments will be described hereinafter in more details. It should be appreciated that, these drawings and the descriptions thereof are not used to limit in any ways the scope of the present disclosure, but are used to describe the present disclosure with reference to the specific embodiments.

### DETAILED DESCRIPTION

In order to make the objects, the technical solutions and the advantages of the present disclosure more apparent, the present disclosure will be described hereinafter in conjunction with the drawings and embodiments.

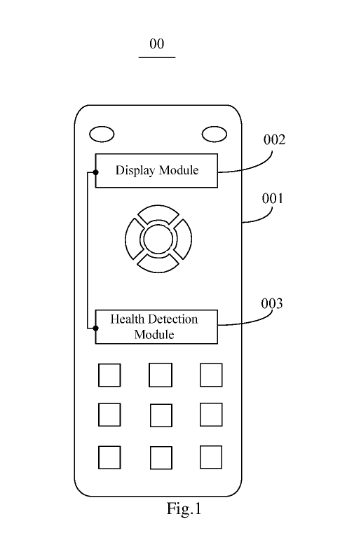

Referring to FIG. 1, the present disclosure provides in some embodiments a remote controller 00, which may include a main body 001, a display module 002 and a health detection module 003 arranged on the main body 001. For example, a plurality of buttons (not shown) may be arranged on the main body 001, and these buttons may be functional ones. FIG. 1 illustratively shows positions where the buttons may be arranged on the main body 001, and these positions may be adjusted in accordance with the practical need.

The display module 002 may be connected to the health detection module 003, the health detection module 003 may be configured to detect a health value of a user, and the health value may at least include a heart rate. The health detection module 003 may, after the health value of the user is detected, transmit the health value to the display module 002. The display module 002 is configured to display the health value of the user upon receiving the health value.

According to the embodiments of the present disclosure, the remote controller includes the main body, the display module and the health detection module arranged on the main body. The health detection module is configured to detect the health value of the user, and the display module is connected to the health detection module and configured to display the health value. When the user wants to detect his health condition, the remote controller may be directly used by the user to detect and display the health value, and the user may know his health condition in accordance with the health value. In this way, it is unnecessary for the user to move the health detection device to be in the vicinity of a television and connect the health detection device to the remote controller. As a result, it is able to simplify the procedure for the health detection.

Further, the display module 002 may be configured to, after receiving the health value of the user, display the health value on the remote controller 00. Alternatively, the display module 002 may also transmit the health value to a television (not shown) after receiving the health value, so as to display the health value on the television. The television matches the remote controller 00, i.e., wireless information transmission may be performed between the remote controller 00 and the television.

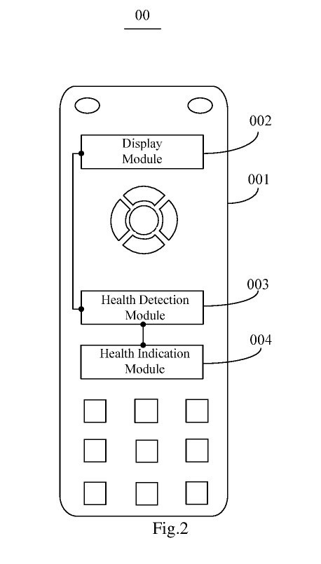

FIG. 2 shows a further remote controller 00 which may detect a health value of the user. The health value may include a heart rate and a blood pressure. The remote controller 00 may include a main body 001, a display module 002, and a health detection module 003 and a health indication module 004 arranged on the remote controller 001.

The display module 002 and the health indication module 004 may be connected to the health detection module 003 which may be configured to detect the health value of the user. The health value may at least include the heart rate. After the health value of the user is detected, the health detection module 003 may transmit the health value to the display module 002, so that the display module 002 displays the health value.

The health indication module 004 is configured to, when the health value detected by the health detection module 003 exceeds a predetermined range, send a prompt. For example, the health indication module 004 may include a lamp (not shown) which may be connected to the health detection module 003. When the health value detected by the health detection module 003 exceeds the predetermined range, the health detection module 003 may generate indication information for indicating the lamp to flicker, and transmit the indication information to the lamp. After receiving the indication information, the lamp may flicker so as to prompt the user to pay attention to this health condition.

The user may use the remote controller 00 to detect his heart rate. For example, there may exist the following ways to detect the heart rate using the remote controller 00.

In a first way, the user may acquire potentials at two different body parts of the user through a portable electrocardiograph device, and calculate a potential difference between the two body parts in accordance with the potentials. There is a correspondence between the potential difference and the heart rate, so it is able to calculate the heart rate of the user in accordance with this correspondence and the potential different between the two different body parts of the user.

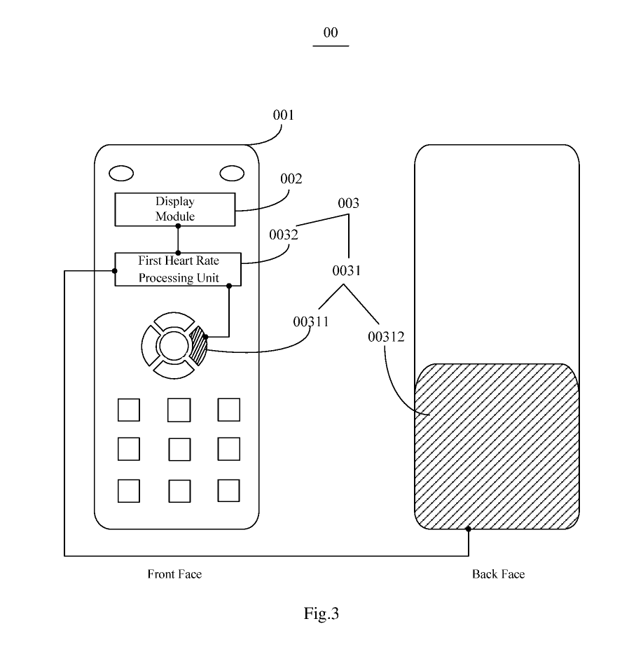

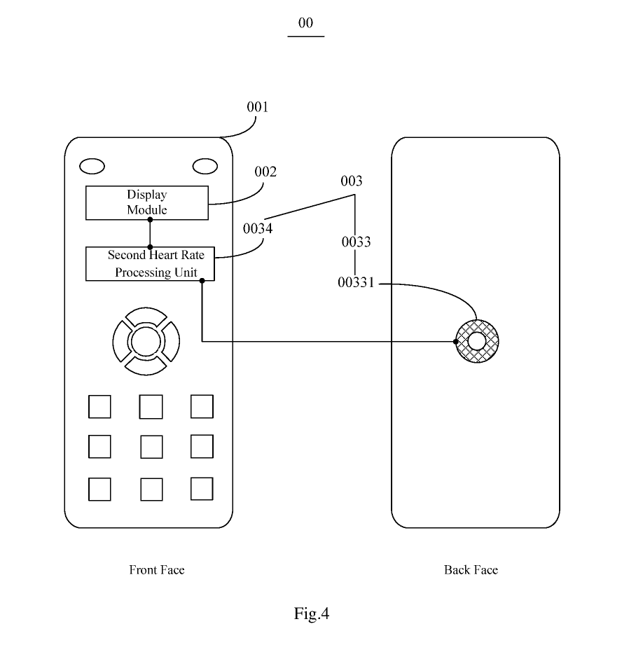

As shown in FIG. 3, which is a schematic view showing the structure of the further remote controller 00 according to one embodiment of the present disclosure, the health detection module 003 may include a first heart rate detection unit 0031 and a first heart rate processing unit 0032 that stores therein the correspondence between the potential difference and the heart rate. The first heart rate detection unit 0031 may include a metal button 00311 and a metal plate 00312. The first heart rate detection unit 0031 is connected to the first heart rate processing unit 0032, and specifically the metal button 00311 and the metal plate 00312 are connected to the first heart rate processing unit 0032. A left portion in FIG. 3 shows a front face of the remote controller 00, and a right portion in FIG. 4 shows a back face of the remote controller 00. The metal button 00311 may be one of a plurality of buttons on the remote controller 00. It should be appreciated that, there may also be a plurality of metal buttons 00311, and the number of the metal buttons is not particularly defined herein. The metal plate 00312 may be arranged at the back face of the remote controller 00, or at any other position on the remote controller 00, i.e., an installation position of the metal plate 00312 is not particularly defined herein.

To be specific, the first heart rate processing unit 0032 is configured to, when a first body part of the user is in contact with the metal button 00311 and a second body part is in contact with the metal plate 00312, acquire a potential at the first body part through the metal button 00311, and acquire a potential at the second body part through the metal plate 00312. Then, the first heart rate processing unit 0032 may calculate a potential difference between the first body part and the second body part in accordance with the potentials at the first body part and the second body part, and calculate the heart rate of the user in accordance with the correspondence between the potential difference and the heart rate as well as the potential difference. The first body part is different from the second body part.

For example, the user may hold the remote controller 00 with his right hand, and touch the metal button 00311 on the remote controller 00 with his left hand, so that the metal plate 00312 is in contact with the user's right hand and the metal button 00311 is in contact with the user's left hand. At this time, the right hand is just the first body part, and the left hand is just the second body part. Then, the first heart rate processing unit 0032 may acquire a potential at the left hand through the metal button 00311, acquire a potential at the right hand through the metal plate 00312, calculate a potential difference between the left hand and the right hand, i.e., between the first body part and the second body part, in accordance with these potentials, and calculate the heart rate of the user in accordance with the correspondence between the potential difference and the heart rate as well as the potential difference.

In a second way, when the heart rate of the user is measured, a skin image of the user may be taken using a camera, so as to acquire a tiny change in colors of vessels and skins when blood flows in the vessels. The colors of the vessels and the skins may be acquired from the skin image, and when the blood flows in the vessels, these colors may change. Hence, the skin image of the user may be acquired by the camera in real time, so as to acquire the tiny change in the colors of the vessels and skin when the blood flows in the vessels, and transmit the tiny change to a computer. The computer may then calculate the heart rate of the user using an image information processing technique and an image-heart rate algorithm.

As shown in FIG. 4, which is another schematic view showing the structure of the further remote controller 00 according to one embodiment of the present disclosure, the health detection module 003 may include a second heart rate detection unit 0033 and a second heart rate processing unit 0034, and the second heart rate detection unit 0033 may include a camera 00331. The second heart rate detection unit 0033 is connected to the second heart rate processing unit 0034. Specifically, the camera 00331 may be connected to the second heart rate processing unit 0034 which may store therein programs related to the image information processing technique and the image-heart rate algorithm. A left portion in FIG. 4 shows a front face of the remote controller 00, and a right portion in FIG. 4 shows a back face of the remote controller 00. The camera 00331 may be arranged at the back face of the remote controller 00, or at any other position on the remote controller 00, i.e., an installation position of the camera 00331 is not particularly defined herein. The camera 00331 is configured to acquire the skin image of the user. To be specific, the camera 00331 may acquire the skin image in real time, and transmit the skin image to the second heart rate processing unit 0034. After receiving the skin image, the second heart rate processing unit 0034 may calculate the heart rate of the user in accordance with the skin image using the image information processing technique and the image-heart rate algorithm.

For example, the user may hold the remote controller 00 with his right hand, so that the camera 00331 at the back face of the remote controller 00 may acquire a skin image of the user's right palm. To be specific, the camera 00331 may acquire the skin image in real time. A flash lamp (not shown) may be arranged in the vicinity of the camera, and when the skin image is to be acquired in a dark state, the flash lamp may be turned on, so that the camera 00331 may acquire a clear skin image. After acquiring the skin image of the right palm, the camera 00331 may transmit the skin image to the second heart rate processing unit 0034, and the second heart rate processing unit 0034 may then calculate the heart rate of the user in accordance with the skin image using the image information processing technique and the image-heart rate algorithm.

In a third way, the user may measure the user's heart rate using a photoelectric cardiotachometer. When the photoelectric cardiotachometer is used, a light beam is emitted by an LED on the photoelectric cardiotachometer, and then reflected by the user's skin toward a phototransistor on the photoelectric cardiotachometer. When the user's heart beats, there is a tiny fluctuation of the vessel, so the light beam from the LED may be reflected by the skin toward a different position of the phototransistor. When this position changes, it means that the user's heart has beaten once, and this heartbeat is recorded by the photoelectric cardiotachometer. The photoelectric cardiotachometer may also record a time period for detecting the heart rate, and then calculate the heart rate of the user in accordance with the time period and the number of heartbeats within the time period.

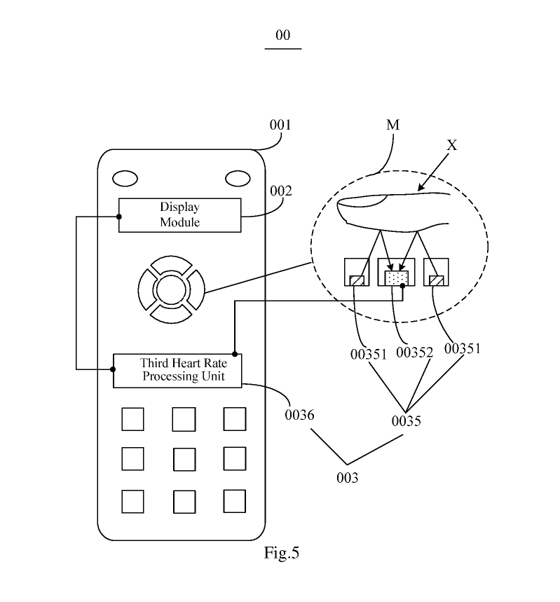

As shown in FIG. 5, which is yet another schematic view showing the further remote controller 00 according to one embodiment of the present disclosure, the health detection module 003 may include a third heart rate detection unit 0035 and a third heart rate processing 0036. The third heart rate detection unit 0035 may include LEDs 00351 and a phototransistor 00352. The LEDs 00351 may be arranged in the vicinity of the phototransistor 00352, and as shown in FIG. 5, there are two LEDs 00351. Of course, one or more than two LEDs 00351 may be provided, i.e., the number of the LEDs is not particularly defined herein. The third heart rate detection unit 0035 is connected to the third heart rate processing unit 0036, and specifically, the phototransistor 00352 is connected to the third heart rate processing unit 0036. Referring to FIG. 5, the LEDs 00351 and the phototransistor 00352 may be arranged within the buttons on the remote controller 00. In FIG. 5, a portion M surrounded by a dotted line shows a working principle of the button in which the LEDs 00351 and the phototransistor 00352 are arranged. For example, the phototransistor 00352 is configured to, when the light beams from the LEDs 00351 are reflected by the user's hand toward the phototransistor 00352, generate detection information in accordance with the reflected light beams, and transmit the detection information to the third heart rate processing unit 0036. Then, the third heart rate processing unit 0036 calculates the heart rate of the user in accordance with the detection information.

To be specific, as shown in FIG. 5, the user may place his finger X onto the phototransistor 00352 and the LEDs 00351. When the light beams from the LEDs 00351 are reflected by the finger X into the phototransistor 00352, the phototransistor 00352 may generate the detection information in accordance with the reflected light beams, and transmit the detection information to the third heart rate processing unit 0036. Then, the third heart rate processing unit 0036 may calculate the heart rate in accordance with the detection information.

The user may also detect a user's blood pressure using the remote controller 00. For example, there may exist the following ways for detecting the blood pressure using the remote controller 00.

In a first way, the user may measure his blood pressure using a non-inflatable hemopiezometer. When the non-inflatable hemopiezometer is used, the user may press a piezoelectric sensor on the non-inflatable hemopiezometer continuously, and a pulse signal generated every time the piezoelectric sensor is pressed is collected by the piezoelectric sensor. Then, the collected pulse signals are transmitted to the non-inflatable hemopiezometer, so that the non-inflatable hemopiezometer may calculate a time difference between the adjacent pulse signals. There is a correspondence between this time difference and the user's blood pressure, so the non-inflatable hemopiezometer may calculate the user's blood pressure in accordance with the correspondence as well as the time difference.

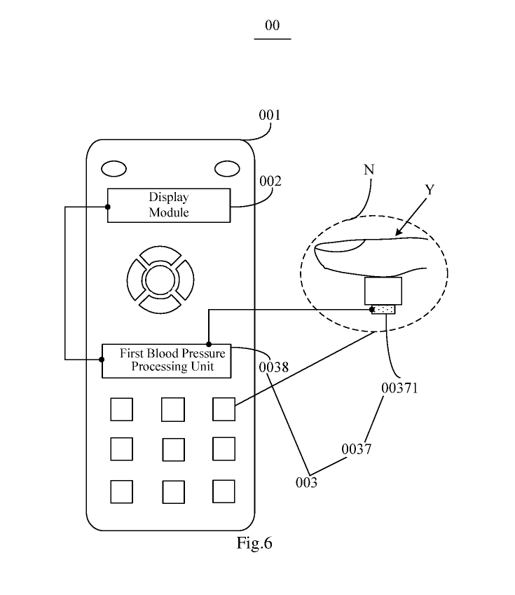

As shown in FIG. 6, which is still yet another schematic view showing the structure of the further remote controller 00 according to one embodiment of the present disclosure, the health detection module 003 may include a first blood pressure detection unit 0037 and a first blood pressure processing unit 0038. The first blood pressure detection unit 0037 may include a piezoelectric sensor 00371, and the first blood pressure processing unit 0038 may store therein the correspondence between the time difference and the blood pressure. The first blood pressure detection unit 0037 is connected to the first blood pressure processing unit 0038, and specifically, the piezoelectric sensor 00371 is connected to the first blood pressure processing unit 0038 and it may be secured beneath a button of the main body 001. In FIG. 6, a portion N surrounded by a dotted line shows a working principle of the button under which the piezoelectric sensor 0371 is arranged. The piezoelectric sensor 00371 may be embedded into a lower portion of the button of the main body 001. When the user presses the button continuously, the piezoelectric sensor 00371 may collect the pulse signal generated every time the button is pressed, and transmit the collected pulse signals to the first blood pressure processing unit 0038. Then, the first blood pressure processing unit 0038 may generate the time difference between the adjacent pulse signals, and calculate the blood pressure in accordance with the correspondence between the time difference and the blood pressure as well as the time difference.

For example, as shown in FIG. 6, the user may place his finger Y onto the button under which the piezoelectric sensor 00371 is arranged, and press the button continuously. When the user presses the button with his finger Y for the first time, the piezoelectric sensor 00371 may collect a user's pulse signal, and transmit the pulse signal to the first blood pressure processing unit 0038. When the user presses the button with his finger Y again, the piezoelectric sensor 00371 may collect the pulse signal again, and transmit the pulse signal to the first blood pressure processing unit 0038. It should be appreciated that, when the user presses the button continuously, a time difference between every two pressing operations is greater than a predetermined value, and this predetermined value may be 0.1 s. After receiving the adjacent pulse signals, the first blood pressure processing unit 0038 may generate the time difference between the adjacent pulse signals, and calculate the user's blood pressure in accordance with the correspondence between the time difference and the blood pressure as well as the time difference.

In a second way, when the blood pressure is to be measured, a skin image of the user may be taken using a camera. Colors of the vessels and the skins of the user may be acquired from the skin image, and when the blood flows in the vessels, these colors may change. Hence, the skin image of the user may be acquired by the camera in real time, so as to acquire the tiny change in the colors of the vessels and skins when the blood flows in the vessels, and transmit the tiny change to a computer. The computer may then calculate the blood pressure of the user using an image information processing technique and an image-blood pressure algorithm.

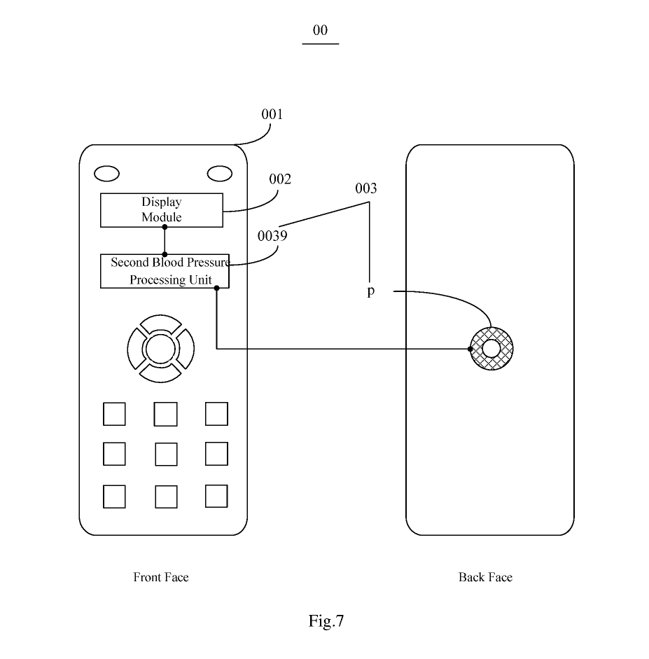

As shown in FIG. 7, which is still yet another schematic view showing the structure of the further remote controller 00 according to one embodiment of the present disclosure, the health detection module 003 may include a second blood pressure processing unit 0039 and a camera P. The camera P may be the camera 00331 in FIG. 4, and the second blood pressure processing unit 0039 may be connected to the camera P and store therein programs related to the image information processing technique and the image-blood pressure algorithm. A left portion in FIG. 7 shows a front face of the remote controller 00, and a right portion in FIG. 7 shows a back face of the remote controller 00. The camera P may be arranged at the back face of the remote controller 00. It should be appreciated that, the camera P may also be arranged at any other position on the remote controller 00, i.e., an installation position of the camera P is not particularly defined herein. The camera P is configured to acquire the skin image of the user. To be specific, the camera P may acquire the skin image in real time, and transmit the skin image to the second blood pressure processing unit 0039, so that the second blood pressure processing unit 0039 calculates the blood pressure of the user in accordance with the skin image using the image information processing technique and the image-blood pressure algorithm.

For example, the user may hold the remote controller 00 with his right hand, so that the camera P at the back face of the remote controller 00 may acquire a skin image of the user's right palm. To be specific, the camera P may acquire the skin image in real time. A flash lamp (not shown) may be arranged in the vicinity of the camera, and when the skin image is to be acquired in a dark state, the flash lamp may be turned on, so that the camera P may acquire a clear skin image. After acquiring the skin image of the right palm, the camera P may transmit the skin image to the second blood pressure processing unit 0039, and the second blood pressure processing unit 0039 may then calculate the blood pressure of the user in accordance with the skin image using the image information processing technique and the image-blood pressure algorithm.

Referring to FIG. 2, after the health value of the user has been detected, the health detection module 003 may transmit the health value to the display module 002, so that the display module 002 displays the health value. It should be appreciated that, the remote controller 00 may communicate with the television (not shown). After receiving the health value, the display module 002 may transmit the health value to the television, so that the television displays the health value. In this way, when watching the television, the user may measure his heart rate and blood pressure using the remote controller 00.

According to the embodiments of the present disclosure, the remote controller includes the main body, the display module and the health detection module arranged on the main body. The health detection module is configured to detect the health value of the user, and the display module is connected to the health detection module and configured to display the health value. When the user wants to detect his health condition, the remote controller may be directly used by the user to detect and display the health value, and the user may know his health condition in accordance with the health value. In this way, it is unnecessary for the user to move the health detection device to be in the vicinity of the television and connect the health detection device to the remote controller. As a result, it is able to simplify the procedure for the health detection.

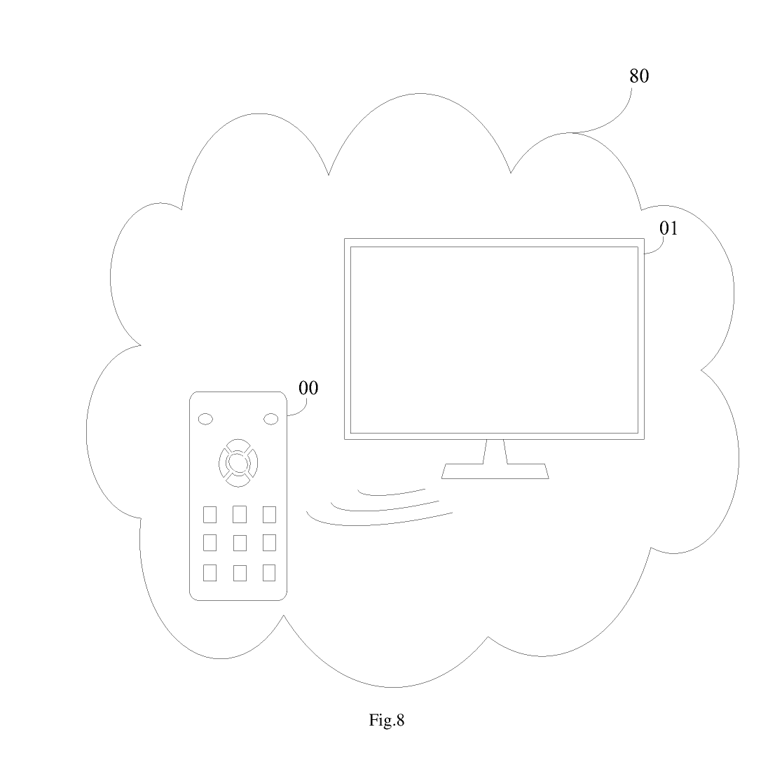

As shown in FIG. 8, the present disclosure provides in some embodiments a health detection system 80, which may include a television 01 and the remote controller 00 in FIGS. 1 to 7.

To be specific, the television 01 may be in wireless communication with the remote controller 00. The remote controller 00 is configured to transmit the detected health value to the television 01, and the television 01 may then display the health value. In addition, the television 01 may also store therein the health value, so that the user may observe his health values within a long period of time and a change in the health values, and adjust his health conditions in accordance with the health values and the change in the health values. For example, the television 01 may store therein the health values, or upload the health values to Cloud.

According to the embodiments of the present disclosure, the health detection system includes the remote controller and the television. The remote controller is configured to detect and display the health value of the user. Also, the remote controller may transmit the health value to the television so that the television displays the health value. When the user wants to detect his health condition, the remote controller may be directly used by the user to detect and display the health value, or the television may be used to display the health value, so that the user may know his health condition in accordance with the health value. In this way, it is unnecessary for the user to move the health detection device to be in the vicinity of the television and connect the health detection device to the remote controller. As a result, it is able to simplify the procedure for the health detection.

The above embodiments may be combined in various forms, and they will not be particularly defined herein.

The above are merely the preferred embodiments of the present disclosure. It should be appreciated that, a person skilled in the art may make further modifications and improvements without departing from the principle of the present disclosure, and these modifications and improvements shall also fall within the scope of the present disclosure.

What is claimed is:

1. A remote controller, comprising a main body, a display module and a health detection module arranged on the main body, wherein
the health detection module is configured to detect a health value of a user, the health value at least comprises a heart rate, and the display module is connected to the health detection module and configured to display the health value.

2. The remote controller according to claim 1, wherein the health detection module comprises a first heart rate detection unit and a first heart rate processing unit connected to the first heart rate detection unit;
the first heart rate detection unit comprises a metal button and a metal plate;
the first heart rate processing unit is configured to, when a first body part of the user is in contact with the metal button and a second body part of the user is in contact with the metal plate, acquire a potential at the first body part through the metal button, acquire a potential at the second body part through the metal plate, calculate a potential difference between the first body part and the second body part in accordance with the potential at the first body part and the potential at the second body part, and calculate the heart value in accordance with a correspondence between the potential difference and the heart rate as well as the potential difference; and
the second body part is different from the first body part.

3. The remote controller according to claim 1, wherein the health detection module comprises a second heart rate detection unit and a second heart rate processing unit connected to the second heart rate detection unit;
the second heart rate detection unit comprises a camera configured to acquire a skin image of the user and transmit the acquired skin image to the second heart rate processing unit; and
the second heart rate processing unit is configured to calculate the heart rate in accordance with the skin image using an image information processing technique and an image-heart rate algorithm.

4. The remote controller according to claim 1, wherein the health detection module comprises a third heart rate detection unit and a third heart rate processing unit connected to the third heart rate detection unit; and
the third heart rate detection unit comprises a light-emitting diode (LED), and a phototransistor configured to, when a light beam from the LED is reflected by a hand of the user to the phototransistor, generate detection information in accordance with the reflected light beam, and transmit the detection information to the third heart rate processing unit so that the third heart rate processing unit generates the heart rate in accordance with the detection information.

5. The remote controller according to claim 4, wherein there is more than one LED.

6. The remote controller according to claim 1, wherein the health value further comprises a blood pressure;
the health detection module comprises a first blood pressure detection unit and a first blood pressure processing unit connected to the first blood pressure detection unit;
the first blood pressure detection unit comprises a piezoelectric sensor secured underneath a button of the main body of the remote controller;
the piezoelectric sensor is configured to, when the button is pressed by the user continuously, collect a pulse signal generated every time the button is pressed, and transmit the collected pulse signal to the first blood pressure processing unit;
the first blood pressure processing unit is configured to generate a time difference between the adjacent pulse signals, and calculate the blood pressure in accordance with a correspondence between the time difference and the blood pressure as well as the time difference.

7. The remote controller according to claim 3, wherein the health value further comprises a blood pressure, and the health detection module further comprises a second blood pressure processing unit connected to the camera; and
the camera is further configured to transmit the skin image to the second blood pressure processing unit, so that the second blood pressure processing unit calculates the blood pressure in accordance with the skin image using the image information processing technique and an image-blood pressure algorithm.

8. The remote controller according to claim 1, further comprising a health indication module connected to the health detection module and configured to, when the health value exceeds a predetermined range, send a prompt.

9. The remote controller according to claim 8, wherein the health indication module comprises a lamp connected to the health detection module and flickering when the health value exceeds the predetermined range.

10. The remote controller according to claim 9, wherein the display module is configured to display the health value on the remote controller, or transmit the health value to a television so as to display the health value on the television.

11. A health detection system, comprising a television and the remote controller according to claim 1.

12. The health detection system according to claim 11, wherein the remote controller is configured to transmit a detected health value to the television, and the television is configured to display the health value.

13. The health detection system according to claim 12, wherein the television is further configured to store therein the health value.

14. The remote controller according to claim 2, further comprising a health indication module connected to the health detection module and configured to, when the health value exceeds a predetermined range, send a prompt.

15. The remote controller according to claim 3, further comprising a health indication module connected to the health detection module and configured to, when the health value exceeds a predetermined range, send a prompt.

16. The remote controller according to claim 4, further comprising a health indication module connected to the health detection module and configured to, when the health value exceeds a predetermined range, send a prompt.

17. The remote controller according to claim 5, further comprising a health indication module connected to the health detection module and configured to, when the health value exceeds a predetermined range, send a prompt.

18. The remote controller according to claim 6, further comprising a health indication module connected to the health detection module and configured to, when the health value exceeds a predetermined range, send a prompt.

19. The remote controller according to claim 7, further comprising a health indication module connected to the health detection module and configured to, when the health value exceeds a predetermined range, send a prompt.

20. The health detection system according to claim 11, wherein the health detection module comprises a first heart rate detection unit and a first heart rate processing unit connected to the first heart rate detection unit;
the first heart rate detection unit comprises a metal button and a metal plate;
the first heart rate processing unit is configured to, when a first body part of the user is in contact with the metal button and a second body part of the user is in contact with the metal plate, acquire a potential at the first body part through the metal button, acquire a potential at the second body part through the metal plate, calculate a potential difference between the first body part and the second body part in accordance with the potential at the first body part and the potential at the second body part, and calculate the heart value in accordance with a correspondence between the potential difference and the heart rate as well as the potential difference; and
the second body part is different from the first body part.
